(19) United States
(12) Patent Application Publication
(10) Pub. No.: US 2025/0319161 A1
(43) Pub. Date: Oct. 16, 2025

(54) IMPROVED METHODS OF TREATING DISEASES RESULTING FROM A MALADAPTED STRESS RESPONSE

(71) Applicant: CORTENE INC., Burlingame, CA (US)

(72) Inventor: Gerard Pereira, Burlingame, CA (US)

(21) Appl. No.: 18/550,798

(22) PCT Filed: Mar. 17, 2022
(86) PCT No.: PCT/US22/71209

§ 371 Date: Sep. 15, 2023

Related U.S. Application Data
(60) Provisional application No. 63/200,609, filed on Mar. 17, 2021.

(51) Int. Cl. A61K 38/22

(52) U.S. Cl. CPC A61K 38/22

(57) ABSTRACT

The present disclosure relates to compositions and methods for treating a maladapted stress response involving the corticotropin-releasing factor receptor subtype 2 (CRFR2), such as myalgic encephalomyelitis/chronic fatigue syndrome or an impairment of the musculoskeletal or the nervous system, resulting in measurable symptom improvement.

### CROSS-REFERENCE TO RELATED APPLICATIONS

This application is a U.S. National Phase Under 35 U.S.C. § 371 of International Application No. PCT/US2022/071209, titled “IMPROVED METHODS OF TREATING DISEASES RESULTING FROM A MALADAPTED STRESS RESPONSE,” filed Mar. 17, 2022, which claims the benefit of and priority to U.S. Provisional Application No. 63/200,609, filed Mar. 17, 2021, the contents of which are incorporated by reference herein in their entirety.

### TECHNICAL FIELD

This disclosure relates to formulations and methods of treating diseases resulting from a maladapted stress response. Certain aspects of the disclosure are directed to formulations and methods of treating myalgic encephalomyelitis/chronic fatigue syndrome.

### BACKGROUND

Previous work considers that myalgic encephalomyelitis/chronic fatigue syndrome (ME/CFS), (also known as systemic exertion intolerance disease, post-viral fatigue syndrome, or chronic fatigue immune dysfunction syndrome), a disease of unknown etiology, whose symptoms and anomalies overlap considerably with a group of diseases often termed functional somatic syndromes (FSS), is associated with the upregulation of the corticotropin-releasing factor receptors (CRFRs, and particularly CRFR2) within the neurons of the raphé nuclei and limbic system.

G protein-coupled receptor (GPCR) internalization, also referred to as receptor- or clathrin-mediated endocytosis, has been widely studied in vitro with variants of CRF receptors. Such in vitro studies show that receptor agonists (such as the endogenous urocortins 1, 2 and 3, or UCN1, UCN2, UCN3, in the case of CRFR2) induce a dose-dependent intracellular signal transduction (measured via cyclic adenosine monophosphate or cAMP, in the case of CRFR2), which is attenuated and/or abolished by pre-exposure to agonists in a manner dependent on agonist potency, agonist concentration, and duration of the pre-exposure.

### SUMMARY

Applicant has recognized an unmet and urgent need for treating diseases resulting from a maladapted stress response, including ME/CFS. Applicant has identified the loss of ability to stimulate the CRF receptors following pre-exposure to result from the internalization, or endocytosis, of the receptor. Therefore, therapeutic formulations and methods of treatment of ME/CFS have been developed that are directed to this mechanism on specific CRF receptors.

Embodiments include methods of treating a corticotropin-releasing factor receptor 2 (CRFR2) maladaptation in a subject in need thereof. One such method includes administering to the subject a CRFR2 agonist in an amount to maintain plasma concentration of the CRFR2 agonist in the subject below a threshold concentration of stimulation (C_{T}) of CRFR2 agonist. In certain embodiments, a persistent improvement in at least one symptom associated with the CRFR2maladaptation occurs in the absence of a concurrent administration of the CRFR2 agonist. Examples of the symptoms associated with the CRFR2 maladaptation include one or more of fatigue, pain, sleep issues, cognitive issues, orthostatic intolerance, body temperature perceptions, flu-like symptoms, headaches or sensory sensitivity, shortness of breath, gastrointestinal issues, urinary issues, musculoskeletal issues, nervous system issues, anxiety, depression, or other characterizations or manifestations of the foregoing. In an instance, extreme fatigue is experienced or characterized as paralysis by a patient. In another instance, an impairment of the musculoskeletal or the nervous system manifests as tremors, ataxia, or dyskinesia. The persistent improvement of one or more of these symptoms can continue for at least 1 week or longer following cessation of the administration of the CRFR2 agonist. The CRFR2 agonist can be one or more of UCN1, UCN2, UCN3, stresscopin-related peptide (SRP), Strescopin (SCP), CT38, CT37, or a pharmaceutically acceptable salt or solvate thereof. In certain embodiments, the CRFR2 agonist contains an acetate salt of CT38 (CT38s). In certain embodiments, CT38s is administered to maintain the plasma concentration below about 0.25 ng/ml (nanograms per milliliter) of CT38 to induce persistent improvement in at least one symptom associated with the CRFR2 maladaptation. In certain embodiments, CT38s can be administered to the subject to achieve an AUC of ˜5 ng·h/ml, which reflects the actual body exposure to CT38 after administration of a dose of the CT38s. In certain embodiments, CT38s can be administered at a rate of at least about 0.0001 μg/kg/h.

In certain embodiments, the CRFR2 maladaptation is myalgic encephalomyelitis/chronic fatigue syndrome (ME/CFS) as determined by the Fukuda Research Case Definition for CFS, the Revised Canadian Consensus Criteria for ME/CFS, or the National Academy of Medicine Clinical Diagnostic Criteria for ME. The CRFR2 maladaptation can be a post-acute sequelae of SARS-CoV-2 infection. The CRFR2 maladaptation can be an impairment of the musculoskeletal or the nervous system, such as Parkinson's disease.

Certain embodiments of treating a CRFR2 maladaptation in a subject in need thereof include administering to the subject a controlled-release dose of a CRFR2 agonist. This controlled-release dose of the CRFR2 agonist is effective to maintain plasma concentrations below a threshold of stimulation of the CRFR2 agonist (C_{T}) and to induce persistent improvement of at least one symptom associated with the CRFR2 maladaptation. Examples of the symptoms associated with the CRFR2 maladaptation include one or more of fatigue, pain, sleep issues, cognitive issues, orthostatic intolerance, body temperature perceptions, flu-like symptoms, headaches or sensory sensitivity, shortness of breath, gastrointestinal issues, urinary issues, musculoskeletal issues, nervous system issues, anxiety, depression, or other characterizations or manifestations of the foregoing. In certain embodiments, the CRFR2 maladaptation is a functional somatic syndrome. In certain embodiments, the functional somatic syndrome is myalgic encephalomyelitis/chronic fatigue syndrome. The CRFR2 maladaptation can be a post-acute sequelae of SARS-CoV-2 infection. In certain embodiments, the persistent improvement comprises improvement in the at least one symptom associated with ME/CFS for at least 1 week or longer following cessation of the administration of the CRFR2 agonist. In certain embodiments, the CRFR2 agonist is one or more of UCN1, UCN2, UCN3, SRP, SCP, CT38, CT37, or a pharmaceutically acceptable salt or solvate thereof. The CRFR2 agonist can contain an acetate salt of CT38 (CT38s). In certain embodiments, the controlled-release dose of the CT38s is administered at a rate not exceeding about 0.03 μg/kg/h. In certain embodiments, CT38s is administered to maintain the plasma concentration below about 0.25 ng/ml of CT38 to induce persistent improvement of the at least one symptom associated with the CRFR2 maladaptation.

Numerous other aspects, features and benefits of the present disclosure may be made apparent from the following detailed description taken together with the drawings. It should be further understood that both the foregoing general description and the following detailed description are exemplary and explanatory and are intended to provide further explanation of the inventions as claimed.

### BRIEF DESCRIPTION OF THE DRAWINGS

Various examples are depicted in the accompanying drawings for illustrative purposes, and should not be interpreted as limiting the scope of the embodiments. Furthermore, various features of different disclosed embodiments may be combined to form additional embodiments, which are part of this disclosure.

FIGS. 1A and 1B are stylized illustrations of the typical behavior of a GPCR agonist on a GPCR. Increasing agonist concentrations increase the output triggered by the GPCR, commencing at C_{T} and continuing until C_{L}, after which output triggered by the GPCR declines, putatively occurring via receptor endocytosis. FIG. 1A illustrates this response in the context of CAMP production from the GPCR in vitro, whereas FIG. 1B illustrates the response in terms of rat heart rate in vivo (such as CRFR2 receptor output) from dosing CT38 (a CRER2-selective agonist).

FIGS. 2A and 2B are graphical representations of the effect of mean CT38 maximum plasma concentration (Cmax) (FIG. 2A) or mean area under the plasma concentration-time curve (AUC) (FIG. 2B) on change in mean maximum heart rate (HRmax) in ME/CFS patients (black line) and healthy subjects (grey line). Shown are plots of mean heart rate change (bpm) versus CT38 Cmax (ng/ml) or AUC (ng·h/ml).

FIGS. 3A and 3B are graphical representations of the effect of mean CT38 Cmax (FIG. 3A) or mean AUC (FIG. 3B) on change in mean minimum diastolic blood pressure (dBPmin) in ME/CFS patients (black line) and healthy subjects (grey line). Shown are plots of mean diastolic blood pressure change (mmHg) versus CT38 Cmax (ng/ml) or AUC (ng·h/ml).

FIG. 4 shows the effect of CT38s on the pre-/post-treatment change in means of total daily symptom score (TDSS) and individual daily symptom score, by dose group (showing relevant p-values), in ME/CFS patients. Shown are bar graphs depicting relative change for TDSS and each symptom in each dose cohort (D01, D03, D06, and D20), emphasizing TDSS or symptom improvement (light grey) and worsening (black).

FIGS. 5A and 5B show the effect of CT38s on the pre-/post-treatment change in the 28-day means (bars), with standard deviations (error bars), of TDSS and individual symptoms scores, for CT38: Cmax<0.25 ng/ml (FIG. 5A); or Cmax>0.25 ng/ml (FIG. 5B), either improving (green) or worsening (purple) with relevant p-values (in italics). Note that scales for TDSS and individual symptoms are different.

FIGS. 6A and 6B are graphical representations of the effect of mean CT38 exposure (total AUC) on the patient-specific pre-/post-treatment change in mean total daily symptom scores (TDSS), stratified by pre-treatment symptom severity and whether achieved Cmax remained below (FIG. 6A) or exceeded (FIG. 6B) C_{T} of CT38 in humans (0.25 ng/ml). Shown are plots of mean TDSS change versus Total CT38 AUC (ng·h/ml), stratified by CT38 Cmax, either less than 0.25 ng/ml (FIG. 6A, green) or greater than 0.25 ng/ml (FIG. 6B, purple), and further stratified by pre-treatment TDSS, either mild (FIG. 6A, open circles) or moderate (FIG. 5A, solid circles).

FIGS. 7A and 7B are graphical representations of the effect of CT38s dosing on the SF-36 physical component score (FIG. 7A) and on the SF-36 mental component score (FIG. 7B), by dose group, in ME/CFS patients. Shown are bar graphs depicting SF-36 score for each dose cohort; error bars indicate standard deviation.

FIGS, 8A and 8B are graphical representations of the effect of CT38s on the means of pre-treatment (purple bars) and post-treatment (green bars), with standard deviations (error bars), of SF-36 physical component score (PCS) (FIGS. 8A and 8B, top panels) and SF-36 mental component score (MCS) (FIGS. 8A and 8B, bottom panels) for CT38s at Cmax<0.25 ng/ml (FIG. 8A) and at Cmax>0.25 ng/ml (FIG. 8B).

### DETAILED DESCRIPTION

Reference will now be made to the exemplary embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the disclosure. Unless defined otherwise, all technical and scientific terms used herein have the same meaning as is commonly understood by one of skill in the art to which this disclosure pertains. All patents and publications referred to herein are incorporated by reference.

As used in the specification and claims, the singular form “a”, “an” and “the” include plural references unless the context clearly dictates otherwise.

As used herein, “treatment” or “treating” or “alleviating” are used interchangeably. These terms refer to an approach for obtaining beneficial or desired results including but not limited to therapeutic benefit. By therapeutic benefit is meant eradication or alleviation of the symptoms or the characterizations or manifestations of the underlying disorder being treated. Also, a therapeutic benefit is achieved with the eradication or alleviation of at least one of the physiological symptoms associated with the underlying disorder such that an improvement is observed in the patient, notwithstanding that the patient may still be afflicted with the underlying disorder.

A “therapeutic effect”, as that term is used herein, encompasses a therapeutic benefit of a treatment as described above.

The term “antagonist”, as used herein, includes any molecule that partially or fully blocks, inhibits, or neutralizes a biological activity of a native polypeptide disclosed herein (e.g., CRFR2). Methods for identifying antagonists of a polypeptide can include contacting a native polypeptide with a candidate antagonist molecule and reducing one or more biological activities normally associated with agonist activity at the native polypeptide.

The term “agonist” is used in the broadest ordinary sense and includes both natural small molecules and peptides as well as synthetic small molecules that partially or fully induce a biological activity of a native polypeptide disclosed herein (e.g., CRFR2). Suitable agonist molecules specifically include native polypeptides, variants of native polypeptides, peptides, small organic molecules, etc. Methods for identifying agonists of a native polypeptide may include contacting a native polypeptide with a candidate agonist molecule and measuring a detectable change in one or more biological activities normally associated with the native polypeptide.

The term “ligand” is used in the broadest sense and includes any molecule that binds to another molecule. For example, both agonists and antagonists of a native polypeptide (e.g., CRFR2) as disclosed herein are ligands of the native polypeptide.

“Activity” for the purposes herein refers to an action or effect of a polypeptide or a synthetic molecule mimicking a polypeptide consistent with that of the corresponding native biologically active protein, wherein “biological activity” refers to an in vitro, in vivo or in human biological function or effect, including but not limited to receptor binding, antagonist activity, agonist activity, or a cellular, biochemical, or physiologic response.

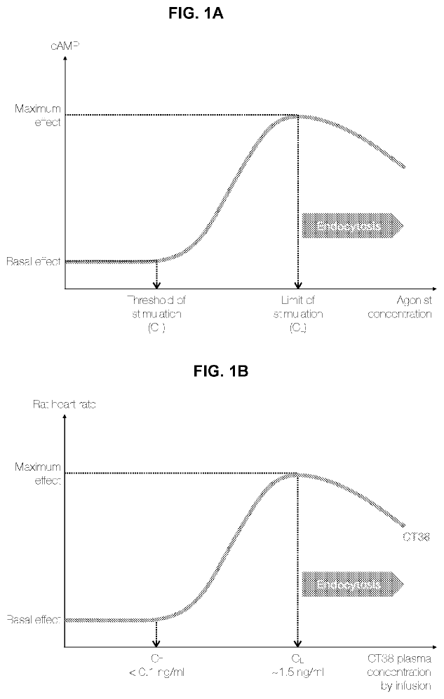

For any agonist, the terms C_{T} (threshold of stimulation) and C_{L} (limit of stimulation) are defined by FIGS. 1A and 1B. In vivo, as shown in FIG. 1B, C_{T} and C_{L} represent the plasma concentrations that invoke an effect at a receptor, and thus C_{L} in particular may vary with the rate at which the agonist is administered (so different for bolus and infused dosing). Cmax (maximum plasma concentration achieved by the drug and AUC (area under the plasma drug concentration-time curve) refer to their standard usage in pharmacology.

A “safe and effective amount” means an amount of the compound (e.g., CRFR2 agonist) according to the disclosure sufficient to induce a significant positive modification in the condition to be treated, but low enough to avoid serious side effects (such as toxicity or irritation) in an animal, preferably a mammal, more preferably a human subject, in need thereof, commensurate with a reasonable benefit/risk ratio when used in the manner of this disclosure. The specific “safe and effective amount” will, obviously, vary with such factors as the particular condition being treated, the physical condition of the subject, the duration of treatment, the nature of concurrent therapy (if any), the specific dosage form to be used, the specific delivery route used, the carrier employed, the solubility of the compound therein, and the dosage regimen for the composition. One skilled in the art may use the following teachings to determine a “safe and effective amount” in accordance with the present disclosure.

The term “pharmaceutically acceptable salt” refers herein to salts derived from a variety of organic and inorganic counter ions and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, “agent” or “biologically active agent” refers herein to a biological, pharmaceutical, or chemical compound or another moiety. Non-limiting examples include a simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present disclosure.

The term “about” generally refers to a plus or minus of 1%, 2%, 3%, 4%, 5%, 6%, 7%, 8%, 9%, 10%, 11%, 12%, 13%, 14%, 15%, 16%, 17%, 18%, 19%, or 20% of the indicated value. For example, about 50 can be interpreted as between 40-60, 43.5-57.5, or 45-55.

As used herein, “subject” refers to an animal, such as a mammal, preferably a human.

As described herein, “controlled-release” refers to any delivery methodology for administering a substance, or a therapeutic drug (e.g., a CRFR2 agonist) to a mammal, including a human, that is intended to maintain the concentration of the agent in the mammal, within a limited range, over some period or periods of time and at a therapeutic level sufficient to achieve a given therapeutic effect. Controlled-release can be continuous-release, time-release, extended-release, sustained-release, delayed-release, prolonged-release, periodic intermittent release, or any combination thereof. A controlled-release could utilize a priming bolus dose in combination with a continuous infusion. A controlled-release could utilize a series of immediate release or bolus doses, provided the concentration of the agent is maintained within a limited range. Controlled-release is effective for maintaining or extending the dissolution, absorption, or administration of the drug to the subject to meet certain parameters for safe and effective treatment (e.g., maintaining a concentration and a duration of dosing with an agent). The substance or therapeutic drug can be a peptide, a drug, or a prodrug described herein. For example, the peptide, drug, or prodrug can be administered via controlled-release using intravenous infusion, subcutaneous infusion, an implantable osmotic pump, subcutaneous depot, a transdermal patch, liposomes, subcutaneous depot injection containing a biodegradable material, or other modes of administration. In some cases, a pump is used. In some cases, polymeric materials are used. In some cases, the flow rate of the peptide, drug, or prodrug is controlled by pressure via a controlled-release system or device. In some cases, a polymer-based drug-delivery system wherein drugs are delivered from polymer or lipid systems. These systems deliver a drug by three general mechanisms: (1) diffusion of the drug species from or through the system; (2) a chemical or enzymatic reaction leading to degradation of the system, or cleavage of the drug from the system; and (3) solvent activation, either through osmosis or swelling of the system. Suitable systems are described in review articles: Langer, Robert, “Drug delivery and targeting,” Nature: 392 (Supp):5-10 (1996); Kumar, Majeti N. V., “Nano and Microparticles as Controlled Drug Delivery Devices,” J Pharm Pharmaceut Sci, 3(2):234-258 (2000); Brannon-Peppas, “Polymers in Controlled Drug Delivery,” Medical Plastics and Biomaterials, (November 1997). See also, Langer, 1990, supra; Treat et al., in Liposomes in the Therapy of Infectious Disease and Cancer, Lopez-Berestein and Fidler (eds.), Liss, New York, pp. 353-365 (1989); Langer, Science, 249:1527-1533 (1990). Suitable systems may include: Atrigel™ drug delivery system from Atrix Labs; DepoFoaM™ from SkyPharma; polyethylene glycol-based hydrogels from Infimed Therapeutics, Inc.; ReGel™, SQZGel™ oral, HySolv™ and ReSolv™ solubilizing drug-delivery systems from MacroMed; ProGelz™ from ProGelz' Products; and ProLease™ injectable from Alkermes.

The phrases “continuous release”, “sustained release”, “sustain release” and “extended release” are used herein to refer to a delivery methodology for administering a substance, or a therapeutic drug, or one or more therapeutic agent(s) that is introduced into the body of a human or other mammal and continuously or continually release or infuse an amount of one or more therapeutic agents over a determined time period and at a therapeutic level sufficient to achieve a given therapeutic effect throughout a determined time period. Reference to a continuous or continual release is intended to encompass release that occurs as the result of biodegradation in vivo of the drug depot, or a matrix or component thereof, or as the result of metabolic transformation or dissolution of the therapeutic agent(s) or conjugates of therapeutic agent(s).

A bolus dose is a delivery methodology for introducing a relatively large quantity of one or more therapeutic agent(s) into the body of a subject via a single rapid administration. In certain embodiments, the therapeutically effective dose can be delivered in the form of a single bolus dose or a series of bolus doses. In an embodiment, a bolus dose of a therapeutic agent can rapidly dissolve or become absorbed at the location to which it is administered. In certain embodiments, a single bolus of a therapeutic agent delivers a controlled-release formulation that releases the therapeutic agent over time to maintain the concentration of the therapeutic agent in a determined range. In these embodiments, the bolus dose is still administered at a single time point and is formulated to delay, prolong, or sustain the introduction, dissolution, or absorption of the therapeutic agent into the body of a subject.

“Functional somatic syndrome” or “FSS” as used herein is meant to indicate a stress-related (or stress-induced) disease resulting from a maladapted stress response associated with a CRFR2 maladaptation (e.g., upregulation of CRFR2) in a certain brain region or regions. Examples of a FSS include, but are not limited to, diseases such as myalgic encephalomyelitis/chronic fatigue syndrome (ME/CFS), fibromyalgia syndrome (FMS), post-traumatic stress disorder (PTSD), irritable bowel syndrome (IBS), atypical depression, multiple chemical sensitivity (MCS), post-acute sequelae of SARS-CoV-2 infection, chronic Lyme disease (CLD), pediatric acute-onset neuropsychiatric syndrome (PANS), pediatric autoimmune neuropsychiatric disorder associated with Streptococcal infections (PANDAS), Gulf War Illness (GWI, sometimes Gulf War Syndrome), non-ulcer dyspepsia, premenstrual syndrome, chronic pelvic pain, interstitial cystitis, low back pain, repetitive strain injury, atypical chest pain, non-cardiac chest pain, hyperventilation syndrome, migraine, tension headache, temporomandibular joint disorder, atypical facial pain, Globus syndrome, food hypersensitivity, and sick building syndrome. An overview of terms and conventional approaches to treatment for functional somatic syndromes can be found, e.g., in Henningsen et al. (Lancet 369(2007): 946-55).

“Dysautonomia” as used herein is meant to indicate a disorder of the autonomic nervous system and is generally characterized by an abnormal heart rate variability, high resting heart rate, inability to alter heart rate with exercise, and exercise intolerance, orthostatic intolerance/hypotension, thermoregulatory intolerance, digestive or urinary abnormalities. Primary dysautonomia is generally considered to be caused by either genetic factors or degenerative neurologic diseases while secondary dysautonomia may occur due to injury or de-regulation of the autonomic nervous system from an acquired disorder.

As used herein, the CRFR2 agonist CT38 may contain free base (CT38) or acetate salt (CT38s) forms. Where pharmacokinetics are reported herein, pharmacokinetics are measured and reported in terms of CT38 (free base).

When reference is made to a composition, pharmaceutical composition or dose amount containing an amount of CRFR2 agonist between about a first ug and about a second ug, the “first μg” term may include the first ug value and the “second μg” term may include the second ug value.

Regulation or treatment of a condition in which CRFR2 is upregulated can benefit a variety of subjects. The subject can be a mammal. In a preferred embodiment, the subject can be a human. In some embodiments, the subject is an adult. In other embodiments, the subject is a child.

Previous work describes the stress-induced interaction between the corticotropin-releasing factor (CRF) system and the serotonin (5HT) system, leading to the downstream modulation of 5HT and its interactions with other neurotransmitters in the brain (e.g., gamma-aminobutyric acid or GABA, glutamate, dopamine, norepinephrine, acetylcholine, histamine) to regulate most body systems. It posits that ME/CFS and other FSSs result from the upregulation of CRFR2 in the neurons of the raphé nuclei and limbic system of the brain. Under such CRFR2 upregulations or maladaptations, low-level stress, which would ordinarily stimulate CRFR1 to release GABA and inhibit 5HT, instead stimulates CRFR2 and increases 5HT release, effectively responding to a minor stress as though it were a major stress.

Such CRFR2 maladaptations and their effect on the stress response, via 5HT, norepinephrine/epinephrine, cortisol (among other mediators) explains virtually all ME/CFS symptoms, including fatigue (increased 5HT in raphé nuclei-spinal pathway inhibits neuronal signals, via 5HT1A), pain (increased 5HT sensitizes dorsal horn via 5HT3A in descending pathways, and increases pain perception in cortical and limbic regions), sleep issues (increased 5HT and norepinephrine promote wakefulness), cognitive impairment (increased norepinephrine promotes reflexive amygdala function over reflective prefrontal cortex function), dysautonomia (increased norepinephrine leads to dysautonomia, which can manifest as heart rate variability, high resting heart rate, inability to alter heart rate with exercise and exercise intolerance, orthostatic intolerance/hypotension, thermoregulatory intolerance, digestive or urinary abnormalities), thermostatic instability (increased 5HT controls temperature, via 5HT2 or 5HT1A, in the preoptic area of the anterior hypothalamus), headaches (5HT implicated in migraine), sensory sensitivity (5HT modulates visual, auditory, olfactory, gustatory and tactile perception), shortness of breath (increased 5HT in the medullary respiratory neurons inhibits breathing), depersonalization (increased 5HT induces transient depersonalization, via 5HT2A or 5HT2C), immune dysfunction (5HT, derived either from platelets or sympathetic neurons that innervate lymphatic tissue, modulates the immune response at inflammatory sites, and can increase systemic susceptibility to infections, allergies and autoimmune disorders), metabolic dysfunction (5HT regulates hypothalamic energy balance, influencing circulating levels of insulin, ghrelin and leptin, and cortisol dysfunction promotes increased plasma insulin and the development of insulin resistance and metabolic syndrome), post-exertional malaise or PEM (5HT is increased during exercise and cognitive effort, thereby exacerbating symptoms), etc. Importantly, such CRFR2 maladaptations are neuronally-specific, and may be present in certain neurons, but not in others. This gives rise to the heterogeneity of symptoms among ME/CFS patients.

CRFR2-5HT maladaptations also explain other characteristics of ME/CFS, including sudden onset (high stress) or gradual onset (cumulative low stress exposure), the variety of triggers (since all provoke the release of CRF and would thus affect CRFR2 within the raphé nuclei, limbic system and/or cortex), post-puberty sex bias (as, relative to males, females may have a heightened stress response mostly through CRFR1- and CRFR2-related mechanisms that emerge at puberty), varied symptom presentation/severity (individual symptoms and their severity respectively result from the precise neurons in which the CRFR2 upregulation exists and the extent of such CRFR2upregulation), risk factors including early life stress or cumulative psychological distress (which would increase CRFR2 upregulation), familial association (which can be genetic or due to similar stress exposure), etc.

Methods of reversal of these CRFR2 maladaptations, such as by chronically blocking or masking the stress response (e.g., via CRF/UCN1/5HT antibodies or 5HT1A/GABA agonists), or by nullifying aberrant expressions of CRFR2 (e.g., via chronically-dosed CRFR2 antagonists) are not appropriate given the highly dynamic nature of these systems. Like most G protein-coupled receptors (GPCRs), CRFR2 is susceptible to intracellular mechanisms that rapidly attenuate signaling output to prevent cell overstimulation. This process has been shown to involve G protein activation and subsequent GPCR kinases (GRKs) regulation, which phosphorylate the receptor and recruit β-arrestin and clathrin to the plasma membrane, leading to receptor endocytosis, at a rate dependent on agonist potency, agonist concentration, and duration of exposure.

Ligand-induced G protein-coupled receptor (GPCR) endocytosis provides a mechanism for alleviating CRFR-related and especially CRFR2-related signaling dysfunctions in the brain. FIGS. 1A and 1B are stylized illustrations of the typical behavior of a GPCR agonist on a GPCR. FIG. 1A is an illustration of the phenomenon, where escalating agonist concentrations, fail to increase cAMP below a threshold of stimulation (“C_{T}”), then increasing CAMP in a dose-dependent manner eventually achieving maximum effect at a limit of stimulation (“C_{L}”), with diminished cAMP response beyond. Dosing regimen/ligand combinations can achieve ligand-induced endocytosis of CRFRs such as CRFR2 to provide therapeutic benefit. Increasing agonist concentrations increase the output triggered by the GPCR, commencing at C_{T} and continuing until C_{L}, after which output triggered by the GPCR declines, putatively occurring via receptor endocytosis.

Previous studies have demonstrated that particular dosing regimens of CRFR2 agonists are able to reduce CRFR2 output through an endocytotic mechanism. For example, WO2018075973A2 demonstrated that increasing concentrations of a proprietary CRFR2 agonist, CT38 (administered as its acetate salt, CT38s), exhibits a dose curve for physiological parameters of rats (e.g., heart rate, mean arterial pressure, and core body temperature) where beyond C_{L} (˜1.5 ng/ml), the capacity of CT38 to induce changes in these physiological parameters diminished (stylized as FIG. 1B, and concordant with the scheme shown in FIG. 1A). Moreover, as heart rate changes under stress have been attributed to CRFR1 and CRFR2 activation in the limbic system (specifically in the bed nucleus of the stria terminalis or BNST), such data indicated CRFR2 endocytosis can occur in the parts of the brain where ME/CFS (and FSS) dysfunction may originate.

These previous experiments, whether in vitro or in vivo, measure endocytosis by the absence of effect and deduce that pronounced endocytosis occurs above C_{L}. For instance, UCN2has been demonstrated to achieve maximal CRFR2 endocytosis in vitro at 100 nmol, which is substantially higher than its EC50 of 4.3 nmol; and CT38 achieves CRFR2 endocytosis in vivo at plasma concentrations greater than C_{L} [see WO2018075973A2, Example 7]. That is, such work demonstrates an endocytotic effect at the upper end of the dose curve. However, no such information has been reported on receptor internalization at concentrations of ligand below C_{T}.

Previous work proposed inducing CRFR2 endocytosis, specifically by utilizing CRFR2 agonists at high concentrations, above C_{L} (FIG. 1B). In contrast, the present disclosure demonstrates endocytosis at different concentration regimes, particularly unexpectedly low concentrations such as those below C_{T} for the agonist (see Example 1).

In a healthy stress response, the release of some level of CRF in the appropriate centers in the brain, results in a given level of stress response. At stressor cessation, the termination of this ongoing stress response appears to rely upon CRFR2 endocytosis, possibly mediated by UCN1 competitively displacing CRF (inhibitory constants for UCN1 and CRF at CRFR2 are 0.4 and 44.5 nmol, respectively). However, to avoid stimulating CRFR2, thereby elevating the very stress response being halted, the level of UCN1 would have to remain below the threshold at which it stimulates CRFR2 (i.e., C_{T} for UCN1). Accordingly, embodiments include administering a CRFR2agonist in conditions where CRFR2 is deemed to be upregulated (or maladapted). In certain embodiments, CRFR2 agonist can reach the site of CRFR2 upregulation (i.e., the raphé nuclei and limbic system), displace CRF (i.e., have a binding affinity for CRFR2 greater than that of CRF), and be administered at a dose that maintains the plasma concentration of the CRFR2 agonist below C_{T} (of the CRFR2 agonist), to induce CRFR2 endocytosis. Without wishing to be limited by theory, such a treatment scheme may involve β-arrestin recruitment without G protein (and/or GRK) activation. Prior reports of CRFR2 endocytosis have been limited to contexts where β-arrestin involvement follows G protein (and/or GRK) activation. Such reports necessitate concentrations above C_{T} and preferentially above C_{L} on the dosing curve.

Embodiments include treatment of any condition involving CRFR2 maladaptations within the raphe nuclei and/or limbic system by administering a CRFR2 agonist, provided the CRFR2 agonist: (i) is capable of reaching the raphé nuclei/limbic system; (ii) is maintained at a concentration below the minimum threshold at which the CRFR2 agonist stimulates CRFR2 (“C_{T}”); and (iii) is administered to provide a certain total exposure (i.e., area under the plasma concentration-time curve or AUC).

In an embodiment, the presence and extent of CRFR2 upregulations (or maladaptations) in a given patient are determined by methods that assess the responsiveness of physiological parameters of a subject to concentrations of a CRFR2 agonist. One such method involves determining the threshold bolus dose of a given CRFR2 agonist in a given patient that is capable of inducing a change in a physiological parameter (such as heart rate, diastolic blood pressure, core body temperature, respiratory rate), and comparing this physiological parameter to a corresponding value for that physiological parameter in healthy subjects (see below), with the difference indicating the presence and extent of CRFR2 maladaptations.

In certain aspects, the compositions and methods disclosed herein are used to treat CRFR2 maladaptations of the stress response that appear in a variety of conditions, including ME/CFS, fibromyalgia syndrome, post-traumatic stress disorder, multiple chemical sensitivities, chronic Lyme disease, post-acute sequelae of viral infections (such as pursuant to a SARS-CoV-2 infection), irritable bowel syndrome, atypical depression, pediatric acute-onset neuropsychiatric syndrome, pediatric autoimmune neuropsychiatric disorder associated with Streptococcal infections, Gulf War Illness, and others (e.g., non-ulcer dyspepsia, premenstrual syndrome, chronic pelvic pain, interstitial cystitis, low back pain, repetitive strain injury, atypical or non-cardiac chest pain, hyperventilation syndrome, migraine, tension headache, temporomandibular joint disorder, atypical facial pain, Globus syndrome, food hypersensitivity, sick building syndrome, etc.), which are understood to represent a single underlying common basic syndrome (FSS, also termed bodily distress syndrome).

In other aspects, the compositions and methods disclosed herein are used to treat dysautonomia (including postural orthostatic tachycardia syndrome, neurocardiogenic syncope, multiple system atrophy, hereditary sensory and autonomic neuropathies, Holmes-Adie syndrome), immune dysfunction (including allergies and autoimmune diseases such as lupus, multiple sclerosis, inflammatory bowel disease, rheumatoid arthritis, type 1 diabetes, celiac disease, and Sjogren's syndrome), Parkinson's disease and other movement disorders, metabolic dysfunction (including insulin resistance, type 2 diabetes, metabolic syndrome), hypothyroidism, hypogonadism. In other aspects, the compositions and methods disclosed herein are used to treat conditions such as chronic pain, anxiety and addiction, which also arise at least in part from CRFR adaptations in the limbic system. In other aspects, the compositions and methods disclosed herein are used to treat an impairment of the musculoskeletal or the nervous system that manifests as tremors, ataxia, or dyskinesia.

For any CRFR2 agonist, its binding affinity at the receptor can be determined, and this can be compared to the binding affinity of CRF at CRFR2 (inhibitory constant=44.5 nmol) to determine whether the CRFR2 agonist is capable of displacing CRF at CRFR2. For instance, the binding affinity of CT38 at CRFR2 (estimated to be similar to that of UCN2, where inhibitory constant=1.1 nmol) is such that it displaces CRF.

In certain embodiments, the CRFR2 agonist is capable of reaching the limbic system, which can be tested, for example, by imaging techniques utilizing radio-labeled agonist in vivo. Alternatively, as the stress response can be mediated by CRFR2, administration of the CRFR2 agonist to healthy animals, alongside observation of significant changes in physiological functions known to be modulated via limbic system CRFR2 (e.g., core body temperature or respiratory rate), can be used to identify suitable CRFR2 agonists. For instance, subcutaneous bolus doses of CT38s induce dose-dependent changes in core body temperature and respiratory rate in laboratory animals, consistent with reaching the limbic system.

In some embodiments, CRFR2 agonists meeting the above criteria are partial agonists or agonists selective for both CRFR2 and CRFR1 (such as UCN1) and are capable of displacing CRF from its receptors in vivo.

In another aspect of this disclosure, different CRFR2 agonists are utilized, where an agonist of higher potency than CT38, will have a lower C_{T}, and may also require adjustments to the target AUC. Specific agonists include CT38 (CT38s), CT37, UCN1, UCN2, UCN3, stresscopin (a putative precursor peptide of UCN3), stresscopin-related peptide (SRP), or any of the sequences described in Table 1. Embodiments set forth here relate to the natural process of endocytosis and are therefore relevant to all CRFR2 agonists, preferentially having a binding affinity for CRFR2 at least greater than that of CRF (for CRFR2). Accordingly, in certain embodiments, the CRFR2 agonist is one or more of the agents presented in Table 1, In some embodiments, the CRFR2 agonist includes one or more of the amino acid sequence of SEQ ID NO: 1, SEQ ID NO:2, SEQ ID NO:3, SEQ ID NO:4, SEQ ID NO:5, SEQ ID NO:6, SEQ ID NO:7, and SEQ ID NO:8, or combinations thereof. In some embodiments, the CRFR2 agonist contains an amino acid sequence having 90%, 91%, 92%, 93% 94%, 95%, 96%, 97%, 98%, 99%, or 100% sequence identity to sequences of SEQ ID NOs. 1-8. In some embodiments, the CRFR2 agonist contains an amino acid to sequence according the formula: ZGPPISIDLPX11X12LLRKX17IEIEKQEKEKQQAX31X32NAX35X36LX38X39X40 (SEQ ID NO: 8) wherein: X11 is selected from F, Y, L, I, and T; X12 is selected from Q, W, and Y; X17 is selected from V and M; X31 is selected from T and A; X32 is selected from N and T; X35 is selected from R and L; X36 is selected from L and I; X38 is selected from D and A; X39 is selected from T and R; X40 is selected from I and V, and wherein Z (i.e., Glx or Pyrrolidone carboxylic acid) is used to indicate N-terminal glutamic acid or glutamine that optionally has formed an internal cyclic lactam. In other embodiments, an acetate salt of the CRFR2 agonist is used.

TABLE 1
Example Peptide Sequences According to the Disclosure

| SEQ ID NO. | Peptide | Amino Acid Sequence |
|---|---|---|
| SEQ ID NO. 1 | CT38 | ZGPPISIDLP FQLLRKVIEI EKQEKEKQQA ANNARLLDTI-NH2 |
| SEQ ID NO. 2 | CT37 | ZGPPISIDLP FQLLRKVIEI EKQEKEKQQA ANNARLLARI-NH2 |
| SEQ ID NO. 3 | Human urocortin 1 | DNPSLSIDLT FHLLRTLLEL ARTQSQRERA EQNRIIFDSV-NH2 |
| | (hUCN1) | |
| SEQ ID NO. 4 | Human urocortin 2 | IVLSLDVPIG LLQILLEQAR ARAARBQATT NARILARV-NH2 |
| | (IUCN2) | |
| SEQ ID NO. 5 | Stresscopin-related | HPGSRIVLSLDVPIG LLQILLEQAR ARAAREQATT NARILARV- |
| | peptide (SRP) | NH2 |
| SEQ ID NO. 6 | Human urocortin 3 | FTLSLDVPTN IMNLLFNIAK AKNLRAQAAA NAHLMAQI-NH2 |
| | (hUCN3) | |
| SEQ ID NO. 7 | Strescopin (SCP) | TKFTLSLDVPTN IMNLLFNIAK AKNLRAQAAA NAHLMAQI-NH2 |
| SEQ ID NO. 8 | | ZGPPISIDLPX₁₁X₁₂LLRKX₁₇IBIEKQEKEKQQAX₃₁X₃₂NAX₃₅X₃₆LX₃₈X₃₉ |
| | | X₄₀ wherein: X₁₁ is selected from F, Y, L, I, and T; |
| | | X₁₂ is selected from Q, W, and Y; X₁₇ is selected |
| | | from V and M; X₃₁ is selected from T and A; X₃₂ is |
| | | selected from N and T; X₃₅ is selected from R and |
| | | L; X₃₆ is selected from L and I; X₃₈ is selected |
| | | from D and A; X₃₉ is selected from T and R; |
| | | X₄₀ is selected from I and V |

In some embodiments, a CRFR2 antagonist is administered to displace CRF, then maintained for a period of time. Such antagonists include, but are not limited to CRFR2-selective Astressin2-B and non-selective Astressin-B.

For any CRFR2 agonist, C_{T} can be determined in healthy animals, including healthy humans. Phase 1 clinical trials routinely identify the profile of an agonist at a receptor. In the case of a CRFR2 agonist, determining its C_{T} at CRFR2 involves administering an escalating bolus dose of the CRFR2 agonist to healthy animals or subjects, measuring the induced change in a physiological parameter associated with CRFR2 stimulation (e.g., heart rate, blood pressure, core body temperature, respiratory rate), and identifying the lowest dose of the CRFR2 agonist that stimulates a significant change in a physiological parameter.

For instance, administered CT38s in healthy human subjects with measurement of physiological parameters, such as heart rate or diastolic blood pressure, identifies the threshold bolus dose of CT38s at CRFR2 in healthy subjects, as between 0.033 (no change in heart rate) and 0.083 μg/kg (average heart rate increase of 8 bpm), equivalent to a plasma concentration of CT38 (free base) of about 0.095 and about 0.250 ng/ml, respectively. Given that the increase in the heart rate at 0.083 μg/kg was small, the C_{T} of CT38s at CRFR2 is estimated to be at or close to 0.25 ng/ml. Individual pharmacokinetics will also affect the estimate of C_{T}, but for all practical purposes a representative range can be determined for a population.

In Example 1, CT38s dosed by subcutaneous infusion at a rate of 0.03 μg/kg/hour (achieving a mean maximum plasma concentration of CT38 of 0.18±0.03 ng/ml; range: 0.14 to 0.24 ng/ml, Table 5), maintained for 3.5 hours over 3 separate treatments (achieving an average total AUC of 2.46±0.51 ng·h/ml; range: 1.94 to 3.44 ng·h/ml), resulted in a mean TDSS improvement of −7.52 (range: −0.3 to −16.0). Notably, while the dose was limited by tolerability with respect to Cmax, it was not limited by AUC. In certain embodiments, for CT38, Cmax cannot exceed 0.25 ng/ml (C_{T}), and total AUC must be ˜5 ng·h/ml for mild symptoms, less for moderate symptoms and even less for severe symptoms. However, as it is normal for patients to have some severe symptoms and some mild, all patients should receive an AUC of ˜5 ng·h/ml, especially as there is no safety issue with providing a higher AUC. Generalizing to any agonist, the Cmax limit is determined by C_{T} (which can be derived for any agonist as above), and the required AUC will be directly related to agonist potency (i.e., a more potent agonist will require less AUC). The precise dosing regimen is then determined by standard pharmacokinetic-pharmacodynamic methods.

In certain embodiments, methods include administering CT38s to a subject having a CRFR2 maladaptation such as ME/CFS, by subcutaneous infusion at a dose level of 0.03 μg/kg/hour, sufficient to ensure a CT38 plasma concentration of no more than 0.25 ng/ml, then maintained for a period of 12 hours to deliver an AUC of ˜2.5 ng·h/ml, and repeated at a second treatment for a total AUC of ˜5.0 ng·h/ml. Alternatively, the same dose level could be maintained for a period of 6 hours to deliver an AUC of ˜1.2 ng·h/ml in each of 5 treatments, for a total AUC of ˜6.0 ng·h/ml.

In some embodiments, CT38s is delivered initially via a subcutaneous priming bolus followed by a subcutaneous infusion, where the priming bolus is intended to shorten the duration of the infusion that would otherwise be required to achieve the target AUC, while maintaining CT38 plasma concentration below 0.25 ng/ml. For example, following a priming bolus of CT38s of 0.03 μg/kg, an infusion at a dose-level of 0.03 μg/kg/hour could deliver an AUC of 1.28 ng·h/ml in each of 4 treatments for a total AUC of ˜5.1 ng·h/ml. The CT38s dosing regimens are designed by taking into account one or more of the use of a priming bolus, the rate of infusion, the duration of infusion, and the number of such treatments. The CT38s dosing regimens are designed to deliver the target AUC (at least ˜5 ng·h/ml) while maintaining CT38 plasma concentration below C_{T} (˜0.25 ng/ml).

In certain embodiments, a series of subcutaneous boluses of a CRFR2 agonist is used to achieve the target exposure, and in such a way that the combined effects of the level and rate of dosing maintain the achieved Cmax below C_{T}. With respect to any agonist having a particular rate of clearance, the rate of infusion determines Cmax and is further affected by the use of a priming bolus. A composition of the disclosure can be administered in a single controlled-release dose, with the total duration of dosing governed by the severity of patient dysfunction at the onset, which bear an inverse relationship (milder severity requires longer duration).

A composition of the disclosure can be administered in multiple controlled-release doses. In such cases, the total duration of dosing is still governed by the severity of patient dysfunction at the onset, and the number of doses and the dosing interval are set by convenience and/or a desire to reassess the level of patient dysfunction before commencing the next dose.

Illustratively, an effective amount of the compositions of this invention ranges from nanograms/kg to micrograms/kg amounts for young children and adults. Equivalent dosages for lighter or heavier body weights can readily be determined. The dose should be adjusted to suit the individual to whom the composition is administered and may vary with age, weight, and metabolic status of the individual. The exact amount of the composition required will vary from subject to subject, depending on the species, age, weight and general condition of the subject, the particular peptide or polypeptide used, its mode of administration and the like. An appropriate amount can be determined by one of ordinary skill in the art using only routine experimentation given the teachings herein. Optimal dosages to be administered may be readily determined by those skilled in the art, and will vary with the particular compound used, the mode of administration, the strength of the preparation, the mode of administration, the number of consecutive administrations within a limited period of time, and the advancement of the disease condition.

Another aspect of the disclosure is a method to determine when to cease treatment in a given patient. In this embodiment, the initially-determined threshold bolus dose of a CRFR2 agonist to induce a heart rate increase in a given patient with a CRFR2 maladaptation increases following treatment as described above. The threshold bolus dose is then redetermined, and when the threshold bolus dose for the patient approximates that of healthy subjects (e.g., about 0.083 μg/kg of body weight of CT38s in healthy young males determined in the prior Phase 1), this indicates that CRFR2 is no longer upregulated in the BNST. Accordingly, in some embodiments, the threshold bolus dose of a CRER2 agonist to induce a heart rate increase is measured in the patient following a treatment, and treatment is stopped when the threshold bolus dose for the patient approximates that of healthy subjects. As it is possible for CRFR2 to remain upregulated in other regions of the brain, which do not affect the heart rate, it may still be appropriate to continue treatment, even after the heart rate suggests a return of CRER2 expression to levels similar to that of healthy subjects.

In addition, as neuron-specific expressions of CRFR2 or 5HT release cannot presently be measured in a live brain undergoing stress, therapeutic effect can be measured via global scales (examples of which include, but are not limited to, the Fatigue Severity Scale, Multidimensional Fatigue Inventory, Patient Global Impression of Change scale, Clinical Global Impression of Change scale, Karnofsky Performance Scale, Fibromyalgia Impact Questionnaire, Short Form-36 or SF-36, Mental Health Inventory, Clinical-Administered PTSD, Inventory of Depression Symptomology, Hamilton Depression Scale, Activities of Daily Living Index, visits to the hospital/emergency room, etc.), or individual symptom scales (examples of which include, but are not limited to, Modified Fatigue Impact Scale, pain visual analog scales, Pittsburgh Sleep Quality Index, American Academy of Sleep Medicine-approved scales for sleep satisfaction quality and improved daytime functioning, Perceived Deficits Questionnaire for cognitive dysfunction, tilt-table tests for orthostatic intolerance, use of concomitant medication, etc.), and ceasing treatment when such scales approximate healthy subject scores.

In certain embodiments, when a CRER2 agonist is administered in such a way to reach the upregulated receptors in the raphé nuclei/limbic system and to be present at a level below the C_{T} of the agonist, CRFR2 endocytosis occurs. However, the extent of CRFR2 maladaptations within the raphe nuclei and limbic system may be variable; therefore, in some embodiments dosage is adjusted for the severity of dysfunction of an individual patient. An example of a case, where treatment adjustment was useful, is partially demonstrated in Example 1 (Table 3 and Table 5), where Patient ID 35 found the relatively low concentration of 0.12 ng/ml difficult to tolerate. To improve treatment tolerability in an individual patient, the dose may be reduced to ensure target plasma concentrations well below C_{T} and the length of administration extended accordingly to provide the appropriate AUC.

In some embodiments, such a reduction in CT38 target plasma concentration is used when a patient displays at least one severe symptom (e.g., pain, sensory sensitivity, brain fog).

In other embodiments, CT38 or any of the CRFR2 agonists described herein (e.g., those described in Table 1) can be delivered by any route of administration, e.g., subcutaneous, intravenous, transdermal, transmucosal, intranasal, inhaled, gastrointestinal etc., to achieve the target plasma concentrations that modulate or reduce a condition associated with a CRER2 maladaptation. Modes of administration of CT38 or any of the CRFR2 agonists described herein can include rectal, oral, buccal, intra-arterial injection, intra-peritoneal, parenteral, intra-muscular, intrathecal, or other appropriate routes.

In other embodiments, the dosage form is formulated for use in treating and/or assessing the severity of an FSS in a subject in need thereof. In some embodiments, the dosage is formulated for subcutaneous administration. In other embodiments, the CRFR2 agonist includes one or more of the amino acid sequences in Table 1. In some embodiments, the CRFR2 agonist includes the amino acid sequence of SEQ ID NO:1, SEQ ID NO:2, SEQ ID NO:3, SEQ ID NO:4, SEQ ID NO: 5, SEQ ID NO:6, SEQ ID NO:7, SEQ ID NO:8, or combinations thereof. In some cases, the polypeptide has a sequence having at least 90%, 91%, 92%, 93% 94%, 95%, 96%, 97%, 98%, 99%, or 100% sequence identity to sequences of SEQ ID NOs. 1-8 as shown in Table 1. In certain embodiments, the controlled-release or bolus dose or dosage form is a pharmaceutical composition containing the protein having the sequence as set forth in Table 1 (e.g., SEQ ID NO: 1) and a pharmaceutically acceptable carrier.

An effective amount of a CRFR2 agonist, or any analogs or derivatives thereof may be administered to a subject according to various dosing regimens. A composition containing the effective amount of a CRFR2 agonist, or any analog or derivatives thereof may be administered in single dose or in more than one dose. The effective amount of a CRFR2 agonist, or any analog or derivatives thereof may be administered in about one dose to about 28 doses, one dose to about 5 doses, or one dose to about 10 doses.

Administration can be initiated at the onset of a disorder or at a time after onset of the disorder. The compositions containing a CRFR2 agonist, or any analogs or derivatives thereof can be administered to a subject who is diagnosed with a CRFR2 maladaptation, or a FSS. In some embodiments, the FSS is myalgic encephalomyelitis/chronic fatigue syndrome (ME/CFS). In other embodiments, the FSS is fibromyalgia syndrome (FMS), post-traumatic stress disorder (PTSD), irritable bowel syndrome (IBS), atypical depression, multiple chemical sensitivity (MCS), chronic Lyme disease (CLD), pediatric acute-onset neuropsychiatric syndrome (PANS), pediatric autoimmune neuropsychiatric disorder associated with Streptococcal infections (PANDAS), or Gulf War Illness (GWI, sometimes Gulf War Syndrome).

In other embodiments, the FSS is a result of an infectious disease and includes a spectrum of symptoms after acute infection. These post-viral symptoms can include post-acute sequelae of SARS-CoV-2 infection (PASC), also referred to as post-coronavirus disease 2019 syndrome or long COVID, whose symptoms overlap considerably with those of ME/CFS. Since 1934, there have been 76 ‘outbreaks’ worldwide, where patients have developed ME/CFS-like symptoms following a pathogenic infection (including viruses, bacteria, mold) and variously described as atypical poliomyelitis (also abortive poliomyelitis, acute anterior poliomyelitis, resembling poliomyelitis, encephalomyelitis associated with poliomyelitis virus), encephalitis, epidemic encephalitis, persistent myalgia, epidemic neuromyasthenia, epidemic myositis, benign myalgic encephalomyelitis, lymphocytic meningo-encephalitis, epidemic malaise, epidemic diencephalomyelitis, infectious venulitis, multi-system stealth virus infection with encephalopathy, chronic fatigue syndrome caused by SARS, chronic fatigue syndrome caused by Giardia, etc.

The composition can be administered to the subject over a period of time. As one example, the composition can be administered to the subject for a period of time from between about 1.0 hour to about 12 hours, 24 hours, or 48 hours. In some embodiments, the composition is administered as a bolus dose of CT38s, or equivalent, of about 0.025 μg/kg, in combination with a continuous infusion dose of about 0.025 μg/kg/hour suitable for a total administration time of about 24 hours. In some embodiments the continuous infusion dose is subdivided, and contains, for instance, multiple continuous infusion doses that together provide the AUC equivalent of about ˜5 ng·h/ml (of CT38). In some embodiments, such a dose combination is additionally adjusted for the severity of the patient's condition.

The composition can be administered to the patient by controlled-release over separate treatment periods. For example, when the C_{T} limit (0.25 ng/ml for CT38) and the required AUC (5 ng·h/ml for CT38) are determined for a therapeutic agent, the dosing regimen determines the duration of treatment. In an embodiment, each of the separate treatment periods are separated by specific time intervals to achieve the desired AUC.

Persons skilled in the art will understand that agonist concentrations, durations of dosing, and intervals of dosing specified in the present disclosure are patient-specific and are also affected by the route of administration and the pharmacokinetic and pharmacodynamic parameters (e.g., potency, half-life) of the specific CRFR2 agonist utilized. For instance, it will be readily apparent that achieved plasma concentrations and AUCs are related, and therefore any reduction in the achieved maximum plasma concentration necessitates an increase in exposure to achieve the target AUC, e.g., via additional boluses or longer duration infusions. It will also be apparent that the achieved plasma concentrations and AUCs will depend on both the dose level of the CRFR2 agonist and the route of administration, which will alter the level of the CRFR2 agonist at the receptor. Routine experiments may be employed to characterize the pharmacokinetics and pharmacodynamics of a CRFR2 agonist dosed via a certain route of administration, and such data would permit conversion of dosing parameters (e.g., dose concentration, treatment length, interval) between different routes of administration. Given knowledge of the specific CRFR2 agonist potency, and the pharmacokinetics for the route of administration dosing models can be constructed to achieve the target plasma concentration. Exemplary information on routes of administration, dosing models, and methods of computation can be found in: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, due to inter-subject variability in both symptom severity and compound pharmacokinetics, individualization of dosing regimen is performed. Dosing for a compound of the disclosure can be found by routine experimentation in light of the instant disclosure.

In another aspect, the present disclosure provides a pharmaceutical composition containing a dosage form of a CRFR2 agonist described herein. The pharmaceutical composition can further contain a pharmaceutical carrier or excipient.

The compositions can be formulated as pharmaceutical compositions to provide an effective amount of a composition containing a CRFR2 agonist. The CRFR2 agonist can be the active ingredient in the formulated pharmaceutical composition.

The disclosure provides pharmaceutical compositions containing (a) an amount of a CRFR2 agonist present that is a safe and effective in treating, reducing and/or alleviating a CRFR2 maladaptation, and (b) a pharmaceutically acceptable carrier.

The forms in which the composition of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

The composition formulated for injection can include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate.

Aqueous solutions in saline are also conventionally used for injection. Aqueous carriers include water, alcoholic/aqueous solutions, emulsions or suspensions, including saline and buffered media. Ethanol, glycerol, propylene glycol, liquid polyethylene glycol, and the like (and suitable mixtures thereof), tromethamine, cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

A composition formulated for injection can include parenteral vehicles, preservatives and other additives. Parenteral vehicles include sodium chloride solution, Ringer's dextrose, dextrose and sodium chloride, lactated Ringer's, or fixed oils. Preservatives and other additives may also be present such as, for example, antimicrobials, anti-oxidants, chelating agents, and inert gases and the like.

Sterile injectable solutions are prepared by incorporating the compound of the present disclosure in the required amount in the appropriate solvent with various other ingredients as enumerated above, as required, followed by filtered sterilization. Generally, dispersions are prepared by incorporating the various sterilized active ingredients into a sterile vehicle, which contains the basic dispersion medium and the required other ingredients from those described herein.

The disclosure also provides kits. The kits include a compound or composition of the present disclosure as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the patient and healthcare provider (such information may include direction for administering an effective amount of a CRFR2 agonist to treat or alleviate a CRFR2 maladaptation such as an FSS. Such information may be based on the results of various studies, for example, studies using experimental animals involving in vivo models and studies based on human clinical trials.

Embodiments of such kits also include a first dosage form of a CRFR2 agonist suitable for administration to a patient in order to detect a CRFR2 maladaptation by a heart rate, blood pressure or other physiological response as described herein. Kits may additionally contain a therapeutic formulation of a CRFR2 agonist appropriate to treat the CRFR2 maladaptation according to the methods described herein. Kits may additionally contain a dosage form of a CRFR2 agonist suitable for administration to a patient to detect a decrease in a CRFR2 maladaptation by a heart rate, blood pressure response or other physiological response following some level of treatment.

In another aspect, the present disclosure also provides kits and articles of manufacture containing materials useful for the treatment and/or diagnosis of disease (e.g., FSS) according to methods described herein. In another embodiment, the disclosure provides kits that include packaging material and at least a first container containing a dosage form or pharmaceutical composition of e.g., a CRFR2 agonist and a label identifying the dosage form or pharmaceutical composition and storage and handling conditions, and a sheet of instructions for the reconstitution and/or administration of the dosage form or pharmaceutical compositions to a subject. In one other embodiment, the kit includes a container and a label, which can be located on the container or associated with the container. The container may be a bottle, vial, syringe, cartridge (including autoinjector cartridges), or any other suitable container, and may be formed from various materials, such as glass or plastic. The container holds a composition having a CRFR2 agonist, an analog or derivative thereof, and can have a sterile access port. Examples of containers include a vial with a stopper that can be pierced by a hypodermic injection needle. The kits may have additional containers that hold various reagents, e.g., diluents, preservatives, and buffers. The label may provide a description of the composition as well as instructions for the intended use.

In one aspect, the present disclosure provides a kit including a container which holds a pharmaceutical composition for administration to a buman patient containing a CRFR2 agonist. In one embodiment, the CRFR2 agonist contains an amino acid sequence having at least about 80%, at least about 85%, at least about 90%, or at least about 95% sequence identity to one of the sequences set forth in Table 1. In another embodiment, the kit further includes a package insert associated with such container. In one other embodiment, the package insert indicates that such composition is for the treatment of ME/CFS or a FSS, by administration of at least one dose of the composition.

Examples included below demonstrate the therapeutic potential of delivering a CRFR2 agonist.

### EXAMPLES

### Example 1—Investigation of the Safety, Tolerability and Efficacy of a Specific CRFR2 Agonist, CT38, in ME/CFS Patients

A single-site, proof-of-concept, open-label Phase 1/2 clinical trial was conducted to evaluate safety, tolerability and efficacy of CT38, administered as the acetate salt (CT38s) by subcutaneous infusion at 1 of 4 dose-levels, in 17 ME/CFS patients. The trial was conducted under a physician-sponsored investigational new drug (IND) application, filed with the US Food and Drug Administration (FDA) and approved by the relevant Institutional Review Board (IRB).

CT38 is a selective CRFR2 agonist with no known off-target activity, except CRFR1 at very high concentrations. It is highly potent for CRFR2 (EC50 nmol/% of Emax: 17.1/100) with similar binding affinity for CRFR2 as UCN2 (inhibitory constant 1.1 nmol). Data from a previous Phase 1 trial (see e.g., WO2018075973A2, which is specifically incorporated herein for the purpose of CT38 pharmacokinetics) indicates CT38 has half-life of ˜1.5 hours.

The Phase 1/2 trial included a recruitment and screening period, followed by enrollment, at least a 4-week pre-treatment assessment period, a 2-week interventional treatment period (involving 3 drug infusions), and at least a 4-week post-treatment assessment period, leading up to exit.

The main aim of the trial was to test whether a limited dose of CT38s could induce persistent CRFR2 endocytosis, which would manifest as reduced (or eliminated) symptoms maintained long after CT38s had been cleared from the body. Accordingly, the trial sought to evaluate: (i) safety, tolerability and pharmacokinetic data following CT38s dosing; (ii) primary efficacy measured as the change in the mean total daily symptom score (TDSS), summed for each of 13 patient-reported daily symptoms (specifically: fatigue, pain, sleep issues, cognitive issues, orthostatic intolerance, abnormal body temperature perceptions, flu-like symptoms, headaches or sensory sensitivities, shortness of breath, gastrointestinal issues, urinary issues, anxiety and depression), each assessed on a 0-5 scale (0=none, 1=very mild, 2=mild, 3=moderate, 4=severe, 5=very severe), and averaged over 28 days before treatment (pre-treatment TDSS) and 28 days before exit from the trial (post-treatment TDSS); and (iii) secondary efficacy via general health status as assessed by the SF-36 physical and mental component scores (PCS and MCS, respectively), each measured on a 0 (maximum disability) to 100 (no disability) scale.

The trial selected male/female ME/CFS patients, aged 18-60, who met the 3 case definitions of ME/CFS (i.e., Fukuda Research Case Definition for CFS, 1994; Revised Canadian Consensus Criteria for ME/CFS, 2010; and the National Academy of Medicine Clinical Diagnostic Criteria for ME/CFS, 2015), had been relatively stable during the preceding 3 months, were willing to comply with study procedures, and provide informed consent. Specific exclusions included active or uncontrolled co-morbidities, pregnancy/breast-feeding, body-mass index>35, cigarette-smoking, substance-abuse, certain medications (including antivirals, antiretrovirals, antibiotics and those interacting with 5HT, norepinephrine, dopamine or cortisol pathways, or a history of severe tachycardia (heart rate>100 bpm), bradycardia (heart rate≤45 bpm), hypotension (rested sitting systolic/diastolic blood pressure<100 mmHg or 60 mmHg, respectively) or renal impairment. There were other minor exclusions, including those related to exercise.

### Dose Evaluation and Selection

WO2018075973A2 identified a C_{L} (by infusion) in rats as ˜1.5 ng/ml, and a total AUC of ˜40 ng·h/ml-equivalent to ˜1.4 ng/ml and ˜7.0 ng-h/ml in healthy humans, based on the pharmacokinetic-pharmacodynamic modeling of the bolus dosing in a prior Phase 1 trial.

The initial treatment regimen proposed 2 separate subcutaneous treatments, each involving an initial priming bolus of 0.15 μg/kg, followed by an infusion of 0.20 μg/kg/hour (escalating to 0.24 μg/kg/hour), over 3 hours. This represented a total dose of 0.825 μg/kg, which was below the maximum tolerated bolus dose in healthy humans of 0.833 μg/kg determined in the prior Phase 1 trial, and projected to achieve a Cmax of 1.4 ng/ml and an AUC of 5.6 ng·h/ml (per treatment), thereby meeting the theoretical targets determined from rats. This was believed to be optimal based on in vivo studies indicating minimization of CRFR2-associated changes in heart rate and blood pressure when dosed by infusion, at the upper end of the dose curve.

The planned initial dose, D20_{pb} (see Table 2) increased heart rate and decreased diastolic blood pressure (more so than systolic) in the first patient, and though this was tolerated (i.e., did not meet the protocol dose-stopping criteria), the level of change made the priming bolus unsafe, which was eliminated in favor of extending the infusion for an extra 30 minutes. Thus, the second treatment in the first patient, and both treatments in the second patient, utilized the D20 dose. The subsequent pharmacokinetic data confirmed that D20_{pb} and D20 (essentially the same total dose) were inducing a level of hemodynamic change that was observed in the prior Phase 1 healthy subjects only at considerably higher concentrations and AUCs. This was the first indication of increased sensitivity to CRFR2 effects in ME/CFS patients, relative to healthy subjects (see below). These findings led to the development of a dose reduction to an infusion rate of 0.03 μg/kg/hour (D03), increase in the dosing duration to 3.5 hours, and addition of a third infusion (while also eliminating the priming bolus and dose rate escalations). To characterize the dose-response curve further, 2 additional infusion rates, 0.06 and 0.01 μg/kg/hour were also examined (D06 and D01, respectively). All dosing changes were approved by the IRB and the FDA. The approved dosing schedule is shown in Table 2.

TABLE 2
Final utilized dosing regimens

| | | |
|---|---|---|
| | Priming | |
| Dose | Bolus | Infusion Rate (μg/kg/h)/Duration (h) |

| Group | (μg/kg) | Initial | Escalation 1 | Escalation 2 |
|---|---|---|---|---|
| D20_{pb} | 0.15 | 0.20/0.75 | 0.22/0.75 | 0.24/1.50 |
| D20 | n/a | 0.20/0.75 | 0.22/0.75 | 0.24/2.00 |
| D06 | n/a | 0.06/3.50 | n/a | n/a |
| D03 | n/a | 0.03/3.50 | n/a | n/a |
| D01 | n/a | 0.01/3.50 | n/a | n/a |

### Patient Disposition and Demographics

In the trial, a total of 17 patients were screened, and 14 enrolled. Of these, 2 patients (IDs 23 and 24) received D20 (or modifications thereof), 2 patients (IDs 34 and 36) received D06, 7 patients (IDs 27, 29, 30, 31, 32, 33 and 46) received D03, and 3 patients (IDs 35, 38 and 45) received D01. During the trial, there were a few unanticipated changes to the approved dosing schedule shown in Table 2, and these are listed in Table 3, along with the explanations. Patient demographics, including demographics broken down by dosing schedules are indicated in Table 4.

TABLE 3
Patient disposition

| Patient | Treatment | Treatment | Treatment | Total Dose | |
|---|---|---|---|---|---|
| IDs | 1 | 2 | 3 | (μg/kg) | Comments |
| 35 | D01 | | | 0.035 | Avoided Treatments 2 and 3 |
| | | | | | due to symptom worsening |
| 38, 45 | D01 | D01 | D01 | 0.105 | |
| 29 | D03 | D03 | D03 | 0.285 | Received 2.5 h at Treatment 1 |
| | | | | | (daylight savings error), |
| | | | | | cannula leaked at Treatment 2, |
| | | | | | so received a 4th infusion |
| 27, 30, 31, | D03 | D03 | D03 | 0.315 | |
| 32, 33, 46 | | | | | |
| 34 | D06 | D06 | | 0.420 | Avoided Treatment 3 for lack |
| | | | | | of venous access |
| 36 | D06 | D06 | D06 | 0.630 | |
| 23 | D20 | D20 | n/a | 0.795 | Cannula leaked at Treatment 1 |
| 24 | D20_{pb} | D20 | n/a | 1.620 | |

* Ignores leaked infusion.

TABLE 4
Patient demographics

| | | D0.01 | D0.03 | D0.06 | D0.20 |
|---|---|---|---|---|---|
| | ALL | (μg/kg/hour) | (μg/kg/hour) | (μg/kg/hour) | (μg/kg/hour) |
| N | 14 | 3 | 7 | 2 | 2 |
| Sex (Male/Female) | 6/8 | 2/1 | 2/5 | 0/2 | 2/0 |
| Race_White | 12 | 3 | 5 | 2 | 2 |
| Race_Other | 2 | 0 | 2 | 0 | 0 |
| Age | 43.7 | 46.0 | 39.7 | 53.6 | 44.6 |
| Age_Onset | 30.8 | 31.0 | 28.6 | 41.5 | 27.5 |
| Age_Diagnosis | 34.6 | 33.7 | 32.4 | 46.0 | 32.5 |
| Onset (Gradual/Sudden) | 8/6 | 2/1 | 3/ 4 | 1/1 | 2/0 |

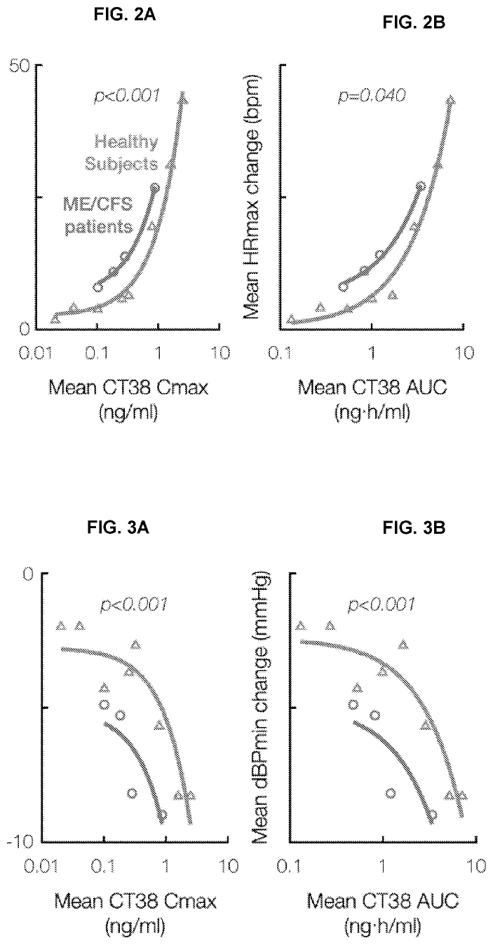

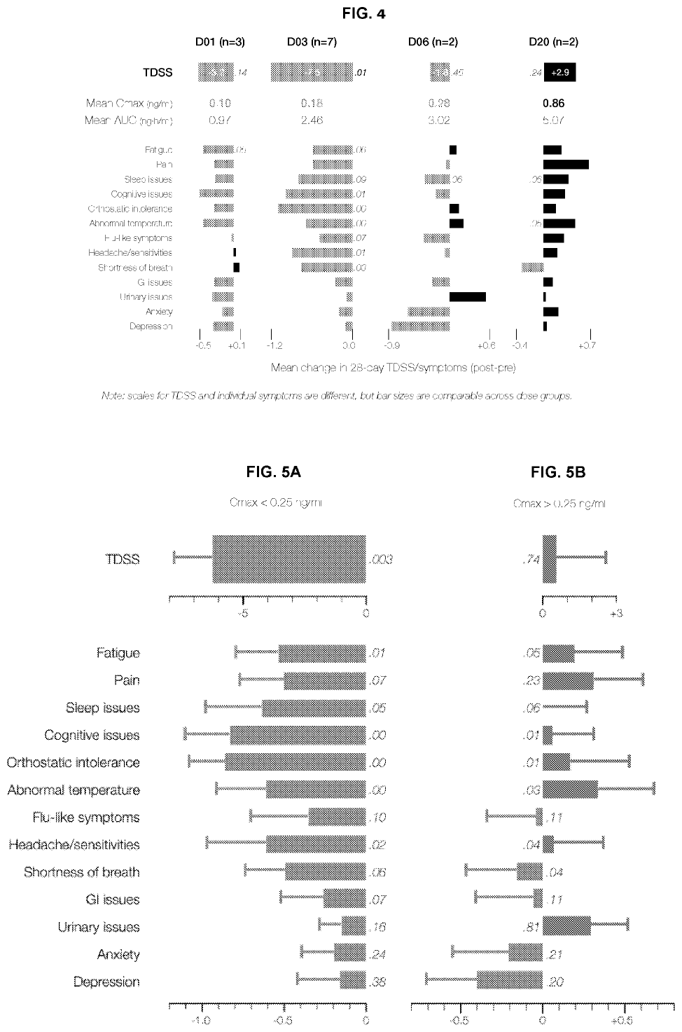

Relative to healthy human subjects in the prior Phase 1 trial, ME/CFS patients displayed increased sensitivity to the acute effects of CRFR2 agonist administration (i.e., increased heart rate and decreased diastolic blood pressure). An example of this phenomenon can be seen in FIGS. 2A-2B and 3A-3B, which show that changes in objectively measured acute heart rate (FIGS. 2A-2B) and diastolic blood pressure (FIGS. 3A-3B) commenced at lower concentrations in ME/CFS patients (0.10 ng/ml) than in healthy subjects (0.25 ng/ml), consistent with CRFR2 upregulation in ME/CFS. FIG. 4 compares the pre/post-treatment changes in TDSS (total daily symptom score) and individual symptoms in the 28-day period prior to the start of treatment, with those in the 28-day period prior to exit from the trial, by dose group. It shows that symptom change was biphasic with dose. TDSS improved persistently at D03 (pre: 29.2±3.7; post: 21.7±4.1; p=0.009; change: −25.7%), and trended better at D01 (pre: 28.7±3.4; post: 25.5±4.1; p=0.136; change: −11.1%) and D06 (pre: 31.3±2.4; post: 29.5±2.1; p=0.451; change: −5.6%), but worsened at D20 (pre: 30.1±5.1; post: 33.0±2.5; p=0.240; change: +9.6%). Most individual symptoms changed persistently, in a manner reflecting this biphasic dose-response. For instance, at D03, the therapeutic response was strong for most of the cardinal symptoms of ME/CFS, while at D20 symptoms predominantly worsened. Individual patient pharmacokinetics and TDSS are indicated in Table 5.

TABLE 5
Patient-reported symptom change, by patient ID

| | Dose | Mean Cₘₐₓ | Total AUC | Mean TDSS ± σ | | | |
|---|---|---|---|---|---|---|---|
| ID | Group | (ng/ml) | (ng · h/ml) | Pre-Rx | Post-Rx | Change | p-value |
| 35 | D01 | 0.12 | 0.59 | 32.4 ± 3.9 | 30.2 ± 3.4 | −2.2 ± 1.0 | 0.038 |
| 38 | D01 | 0.09 | 1.29 | 38.6 ± 3.3 | 32.8 ± 5.4 | −5.8 ± 1.2 | <0.001 |
| 45 | D01 | 0.11 | 1.02 | 15.0 ± 2.9 | 13.5 ± 3.1 | −1.6 ± 0.8 | 0.031 |
| 27 | D03 | 0.24 | 3.44 | 24.9 ± 3.6 | 14.2 ± 3.9 | −10.8 ± 1.1 | <0.001 |
| 29 | D03 | 0.16 | 2.23* | 43.2 ± 2.8 | 27.2 ± 1.6 | −16.0 ± 0.6 | <0.001 |
| 30 | D03 | 0.20 | 2.83 | 13.8 ± 4.2 | 7.4 ± 3.8 | −6.3 ± 1.1 | <0.001 |
| 31 | D03 | 0.17 | 2.19 | 44.7 ± 3.3 | 40.2 ± 2.2 | −4.4 ± 0.8 | <0.001 |
| 32 | D03 | 0.16 | 2.30 | 23.8 ± 3.8 | 19.4 ± 4.6 | −4.4 ± 1.1 | 0.003 |
| 33 | D03 | 0.17 | 2.29 | 21.9 ± 4.8 | 21.6 ± 6.5 | −0.3 ± 1.5 | 0.871 |
| 46 | D03 | 0.15 | 1.94 | 32.5 ± 2.8 | 22.1 ± 4.1 | −10.4 ± 0.9 | <0.001 |
| 34 | D06 | 0.31 | 2.94 | 30.3 ± 2.5 | 27.1 ± 2.4 | −3.3 ± 0.7 | <0.001 |
| 36 | D06 | 0.27 | 3.10 | 32.3 ± 2.3 | 32.0 ± 1.8 | −0.3 ± 0.6 | 0.694 |
| 23 | D20 | 1.03 | 3.91* | 24.5 ± 5.3 | 26.3 ± 3.4 | 1.8 ± 1.5 | 0.351 |
| 24 | D20 | 0.77 | 6.24 | 35.8 ± 4.9 | 39.8 ± 2.1 | 4.0 ± 1.0 | 0.001 |

*Ignores leaked infusion; TDSS = total daily symptom score (patient reported).

Previous work describing CRFR2 endocytosis as a therapeutic approach assumed, based on diminished ability of CT38 to stimulate a heart rate increase in rats subjected to concentrations higher than C_{L} (1.5 ng/ml in a rat; human equivalent ˜1.4 ng/ml), that CRFR2 endocytosis occurred predominately within this range (stylized as FIG. 1B). This trial intended to replicate the rat studies in humans, but found concentrations above ˜0.7 ng/ml to be intolerable.

Surprisingly however, at D01 and D03 where Cmax remained below 0.25 ng/ml, identified as the C_{T} for CT38s in healthy human subjects in the prior Phase 1 trial, there was a long-lasting symptom improvement in ME/CFS patients (FIG. 4). Moreover, as CT38 only binds to CRFR2, and as the observed symptoms were evident at least 28 days after the last treatment with a peptide whose half-life is ˜1.5 hours and would have been long cleared, this suggests that CRFR2 endocytosis may have occurred. This is consistent with UCN1 terminating the stress response by bringing about CRFR2 endocytosis at low concentrations, as discussed above. It is also consistent with a mechanism where, optimal CT38-induced endocytosis occurs in the raphé nuclei/limbic system involving both concentrations of CT38 below its C_{T} (˜0.25 ng/ml).

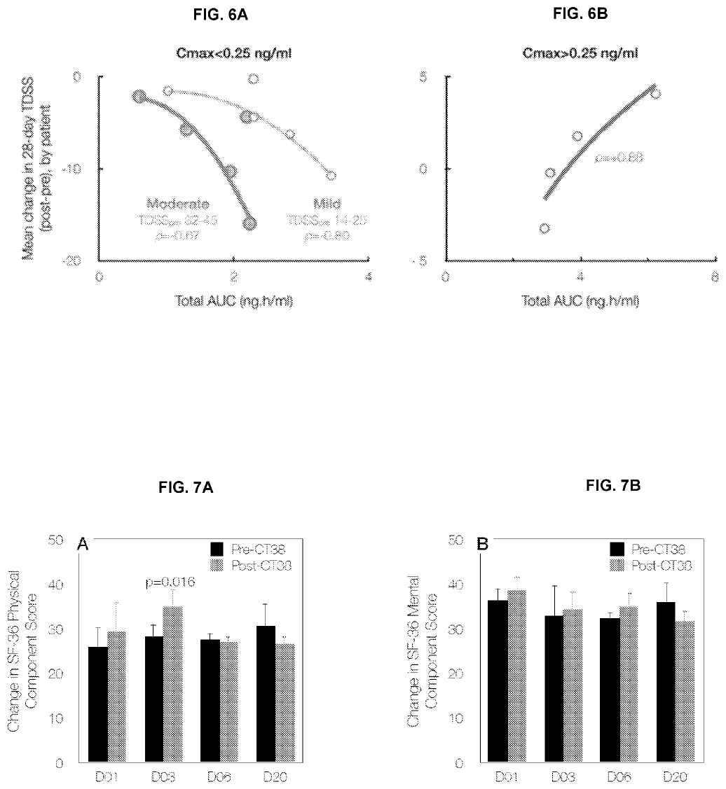

Accordingly, stratifying for a Cmax threshold of 0.25 ng/ml (beyond which symptom improvement declined), the 28-day mean TDSS improved significantly for Cmax<0.25 ng/ml (n=10, TDSSₚᵣₑ: 29.1±3.6, TDSSₚₒₛₜ: 22.9±4.1, p=0.003, change: −6.2±1.6 or −21.4%), with all individual symptoms improving and several achieving significances (FIG. 5A). The improvement in 28-day mean TDSS correlated directly with AUC and indirectly with pre-treatment symptom severity (FIG. 6A, respective Pearson's correlation coefficients: −0.67 and −0.80 for patients with moderate symptoms, TDSSₚᵣₑ=32-45, or mild symptoms, TDSSₚᵣₑ=14-25). For Cmax>0.25 ng/ml, the 28-day mean TDSS worsened though not significantly (FIG. 5B, n=4, TDSSₚᵣₑ: 30.7±4.0, TDSSₚₒₛₜ: 31.3±2.3, p=0.740, change: +0.6±2.0 or ±1.8%), but this lack of significance resulted from different AUCs, as there was a strong direct correlation between 28-day mean TDSS change and AUC (FIG. 6B, Pearson's correlation coefficient: +0.88).

FIGS. 6A and 6B show the persistent changes in patient-specific TDSS to be dose-dependent with CT38 exposure (total AUC). However, for Cmax less than 0.25 ng/ml (D01 and D03), symptoms improved, and were inversely correlated with pre-treatment symptom severity (FIG. 6A with Pearson's correlation coefficients of −0.80 for mild symptoms, TDSSpre=14-25, and −0.67 for moderate symptoms, TDSSpre=32-45). This is consistent with endocytosis occurring below 0.25 ng/ml, and the inverse relationship with pre-treatment symptom severity is consistent with the view of endocytosis as protective against overstimulation. That is mild patients, with only mildly stimulated receptors pre-treatment, need relatively more drug to achieve overstimulation, than moderate patients with moderately stimulated receptors pre-treatment. For Cmax greater than 0.25 ng/ml (D06 and D20), where CRFR2 stimulation would have occurred symptoms worsened (FIG. 6B, Pearson's correlation coefficient=+0.88). Note that at D06, mean Cmax ranged from 0.27 to 0.31 ng/ml, which though above 0.25 ng/ml, would have been below from most of the infusion, and thus TDSS improved, but less so than at D03. Extrapolation of FIG. 6A suggests that subject to remaining below 0.25 ng/ml, mild symptoms (max TDSS 25) and moderate symptoms (max TDSS 45) are respectively eliminated by total AUCs of ˜4.9 and 3.7 ng·h/ml.

The observation that CT38 does not increase heart rate below C_{T} (0.25 ng/ml) in healthy subjects, and therefore does not activate G proteins and second messengers, and yet appears to do so at 0.10 ng/ml in ME/CFS patients (FIG. 2A), is consistent with elevated constitutive (i.e., agonist-independent) activity, putatively arising from increased levels of CRFR2 expression. Note also that the observed CRFR2 sensitivity (FIGS. 2A-2B and FIGS. 3A-3B) has potential use as a diagnostic for ME/CFS, where a given dose of CT38 will induce a change in heart rate or blood pressure (or other physiological parameter) that can be compared to a reference standard in healthy subjects to determine the extent of CRFR2 upregulation in a given ME/CFS patient).

### Results—Patient-Assessed Survey Instruments

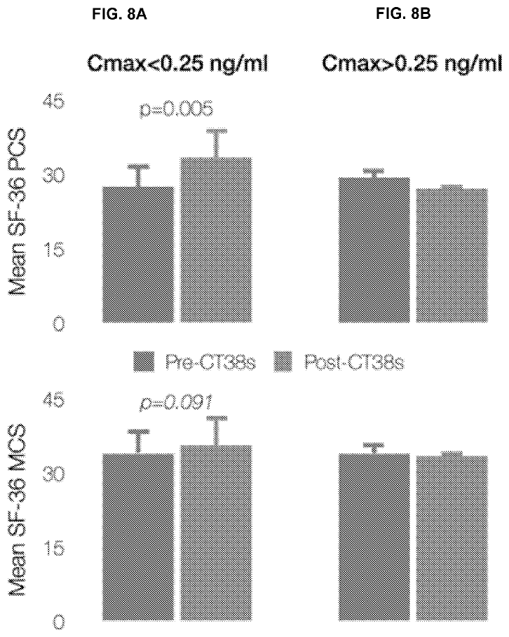

FIGS. 7A-7B show the SF-36 physical (FIG. 7A) and mental (FIG. 7B) component scores for the 4-week periods prior to treatment (pre) and prior to exit from the trial (post). The pre-treatment general health of the patients (PCS: 25.9-30.6; MCS: 25.7-38.1) indicated more impaired health than national averages for cancer (PCS=45.1; MCS=48.8), congestive heart failure (PCS=31.0; MCS=45.7) or diabetes (PCS=39.3; MCS=47.9). The SF-36 score trends show the same biphasic character, improving at D03 (significantly for PCS, p=0.016) and D01, worsening at D20, and displaying intermediate values for D06. Note that the concurrence of the non-validated TDSS with the highly validated SF-36 endpoint (albeit not specifically in ME/CFS), lends support to the former. Another way of looking at the data, FIGS. 8A and 8B are graphical representations of the effect of CT38s on the means of pre-treatment (purple bars) and post-treatment (green bars), with standard deviations (error bars), of SF-36 physical component score (PCS) (FIGS. 8A and 8B, top panels) and SF-36 mental component score (MCS) (FIGS. 8A and 8B, bottom panels) for CT38s at Cmax<0.25 ng/ml (FIG. 8A) and at Cmax>0.25 ng/ml (FIG. 8B).

Tolerability and symptom worsening at concentrations above 0.25 ng/ml necessitated a reduced infusion rate, so limiting the AUC that could be delivered in the allotted timeframes. However, provided Cmax remained below 0.25 ng/ml, safety concerns were absent, and thus longer/additional infusions, to bring exposure closer to target (respectively, 3.7 and 4.9 ng·h/ml for moderate and mild symptoms by extrapolation, FIG. 6A), should increase efficacy. This aspect was validated in five patients (D03), who received 3-4 treatments sufficiently-separated to assess the effect of each treatment, resulting in mean TDSS decreases following treatments 1 and 2 combined (too close to separate), 3 and 4 (ID29). Overall, the data support the notion that the AUC can be delivered as a single or multiple treatments, and increasing AUC increases effect.

### Results—Safety Assessment

Most treatment-emergent adverse events (TEAEs) were mild (mild 79.0%; moderate 18.5%; severe 2.5%), predominantly acute in onset (within 10 hours of dose commencement) and resolved without intervention (Table 6). The only severe TEAE occurred at the highest dose (D20), involving tachycardia and hypotension (treated with intravenous saline) in 1 patient, partially related to an inadvertent protocol deviation that failed to stop dosing, despite the dose-stopping criteria being met. With the exception of acute cardiovascular changes and flushing (known to involve CRFR2-induced peripheral vasodilation) occurring during the infusions, TEAEs were similar to those before treatment and largely indistinguishable from the symptoms of ME/CFS in general, consistent with the proposed mechanism of action.

TABLE 6
Treatment-Emergent Adverse Events (TEAE), by Dose Group

| | | D0.01 | | D0.03 | | D0.06 | | D = 0.20 | | |
|---|---|---|---|---|---|---|---|---|---|---|
| | | E = 7 | | E = 22 | | E = 5 | | E = 4 | | |
| TEAEs | | Mild | Moderate | Mild | Moderate | Mild | Moderate | Mild | Moderate | Severe |
| Total | | 30 | 6 | 69 | 6 | 16 | 7 | 9 | 10 | 4 |
| Treatment Day | Flushing | 7 | | 20 | 1 | 1 | 4 | 2 | 2 | |
| | Cardiovascular | | | 4 | 1 | 2 | | 2 | 1 | 2 |
| TDSS-Related | Headaches/sensitivities | 5 | 2 | 7 | 1 | 2 | 1 | 1 | | |
| | Fatigue | | | 7 | | 2 | | 1 | 2 | 2 |
| | Flu-like symptoms | 2 | | 8 | 1 | 1 | | | 1 | |
| | Abnormal temperature | 5 | | 3 | 1 | 1 | | 1 | | |
| | sensations | | | | | | | | | |
| | GI symptoms | | 1 | 4 | 1 | 2 | | 1 | | |
| | Sleep issues | 1 | | 5 | | | | | 1 | |
| | OI symptoms | 2 | 1 | 2 | | | | | 1 | |
| | Muscle/joint pain | 1 | | 2 | | | 1 | | | |
| | Cognitive symptoms | 2 | | 1 | | | | | | |
| | Shortness of breath | 1 | 1 | 1 | | | | | | |
| | Depression | | | | | | | | 2 | |
| | Anxiety | | | | | | | 1 | | |
| Other | Cardiovascular | | 1 | | | 2 | 1 | | | |
| | Psychological | 2 | | 1 | | 2 | | | | |
| | Neurological | 2 | | 1 | | 1 | | | | |
| | Pain | | | 2 | | | | | | |
| | Metabolism/nutritional | | | 1 | | | | | | |

Data indicate the actual number of recorded events;
E = total number of drug exposures, by dose group

In the long-term follow-up of 9 (of 14) patients, who were regular patients of the principal investigator, the effects appear to be holding over a year from treatment (and close to 2 years in the earlier-treated patients). Patient narratives noted subtle improvements in sleep, cognition, appetite and activity. They observed that the character of their post-exertional malaise had changed, in that they crashed less often and recovered more quickly from a crash, but did not appreciate these improvements until weeks/month after the final dose. This is consistent with a perturbed system, with a multitude of imbalanced effector proteins (e.g., cytokines, hormones, etc.) and downstream effects, where CRFR2 down-regulation removes the underlying sensitivity to perturbation, not the effector proteins, which take longer to metabolize.

Table 7 captures this long-term data. It tracks ‘hours of upright activity’ (HUA), which is a measure used at the principal investigator's clinic to gauge function. Several patients show improvements in this metric. In addition, the principal investigator provided an assessment of the patients, showing that 6 patients improved, likely due to CT38 treatment. Moreover, it should be noted that the AUCs delivered (Table 7) were relatively low compared to target of ˜5 ng·h/ml, determined in FIG. 6A, but as noted above, nothing prevents increasing the AUC (provided the concentration of the agonist is maintained below the threshold at which it stimulates CRFR2). These data support a long-term effect from an essentially ‘one-time’ dose, i.e., potentially a cure.

TABLE 7
Long-term follow-up in a subset of patients

| | Dose | Total AUC | | | | |
|---|---|---|---|---|---|---|
| ID | Group | (ng · h/ml) | Pre-Rx | Post-Rx | Improvements | PI Assessment |
| 38 | D01 | 1.29 | November 2018 | March 2019 HUA 10-14 | Mild cognitive | |
| | | | HUA 10-14, | July 2019 HUA 10 | | |
| | | | works but no | | | |
| | | | exercise tolerance | | | |
| 45 | D01 | 1.02 | August 2018 | April 2019 HUA 2-4 | More good days, | Improved, but |
| | | | HUA 2-4 | October 2019 HUA 4 | activity tolerance, | many variables |
| | | | | January 2020 HUA 5 | fatigue, sleep, | due to other |
| | | | | on pyridostigmine, | appetite | treatments |
| | | | | desmopressin, valacyclovir, | | |
| | | | | still has limits and can | | |
| | | | | easily trigger PEM | | |
| 27 | D03 | 3.44 | | | Sleep, cognition, OI, | Improved |
| | | | | | less wired/lower drive, | |
| | | | | | lower resting HR | |
| 29 | D03 | 2.23* | June 2018 | April 2019 HUA 12-14 | “Small improvements”, | Improved: |
| | | | HUA 5 | | OI | Completed CNA |
| | | | | | | training and |
| | | | | | | began working |
| | | | | | | Fall 2019 |
| 30 | D03 | 2.83 | July 2018 | March 2019 HUA 6 | “A little more energy”, | |
| | | | HUA 3-6 | July 2019 HUA 12 | activity tolerance, OI, | |
| | | | | after valacyclovir | PEM mostly in first few | |
| | | | | and hydroxychloroquine | months | |
| | | | | September 2019 | | |
| | | | | HUA 6 Part time | | |
| | | | | job + school too much | | |
| | | | | January 2020 HUA 7 | | |
| | | | | PT work & travel | | |
| 32 | D03 | 2.30 | March 2018 | July 2019 HUA 11 | Activity tolerance, | Improved |
| | | | HUA 6-8 | | sleep, QOL, cognition, | |
| | | | | | slow/steady | |
| | | | | | improvement | |
| 33 | D03 | 2.29 | July 2018 | February 2019 HUA 6 | OI, needs less sleep to | Improved |
| | | | HUA 6 | August 2019 HUA 6 | recover, managing life | |
| | | | | | stress better | |
| 46 | D03 | 1.94 | July 2018 | March 2019 HUA 7 | Activity tolerance, OI, | Improved |
| | | | HUA 1 (one) | August 2019 HUA 10 | headaches, exercise | |
| | | | | able to hike | tolerance, cognition | |
| 36 | D06 | 3.10 | August 2018 | May 2019 HUA 11 | Activity tolerance and | Improved |
| | | | HUA 9-13 | new breast cancer | cognition definitely | |
| | | | | dx - - - lump + XRT | improved which helped | |
| | | | | January 2020 HUA II | her get through a cancer | |
| | | | | | diagnosis, “new | |
| | | | | | plateau” | |

*Ignores leaked infusion; HUA = average daily Hours of Upright Activity (patient reported).

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein. To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

It is to be understood that the words which have been used are words of description rather than limitation, and that changes may be made within the purview of the appended claims without departing from the true scope and spirit of the embodiments in its broader aspects. While the embodiments have been described at some length and with some particularity with respect to certain aspects, it is not intended that the disclosure should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A method of treating a corticotropin-releasing factor receptor 2 (CRFR2) maladaptation in a subject in need thereof, comprising administering to the subject a CRFR2 agonist in an amount to maintain plasma concentration of the CRFR2 agonist in the subject below a threshold concentration of stimulation (C_{T}) of CRFR2 agonist.

2. The method of claim 1, wherein a persistent improvement in at least one symptom associated with the CRFR2 maladaptation occurs in the absence of concurrent administration of the CRFR2 agonist.

3. The method of claim 2, wherein the persistent improvement continues for at least 1 week or longer following cessation of the administration of the CRFR2 agonist.

4. The method of claim 1, wherein the CRFR2 agonist is one of an urocortin peptide (UCN1, UCN2, or UCN3), stresscopin-related peptide, strescopin, CT38, CT37, or a pharmaceutically acceptable salt or solvate thereof.

5. The method of claim 1, wherein the CRFR2 agonist contains an acetate salt of CT38 (CT38s).

6. The method of claim 5, wherein the CT38s is administered to the subject to achieve an area under the plasma concentration-time curve (AUC) of ˜5 ng·h/ml.

7. The method of claim 5, wherein the CT38s is administered to maintain the plasma concentration below about 0.25 ng/ml of CT38s to induce persistent improvement in at least one symptom associated with the CRFR2 maladaptation.

8. The method of claim 5, wherein the CT38s is administered at a rate of at least about 0.0001 μg/kg/h.

9. The method of claim 1, wherein the CRFR2 maladaptation is myalgic encephalomyelitis/chronic fatigue syndrome (ME/CFS) as determined by the Fukuda Research Case Definition for CFS, the Revised Canadian Consensus Criteria for ME/CFS, or the National Academy of Medicine Clinical Diagnostic Criteria for ME.

10. The method of claim 1, wherein the CRFR2 maladaptation is a post-acute sequelae of SARS-CoV-2 infection.

11. A method of treating a CRFR2 maladaptation in a subject in need thereof, comprising administering to the subject a controlled-release dose of a CRFR2 agonist, wherein the controlled-release dose of the CRFR2 agonist is effective to maintain plasma concentrations below a threshold of stimulation of the CRFR2 agonist (C_{T}) and to induce persistent improvement of at least one symptom associated with the CRFR2 maladaptation.

12. The method of claim 11, wherein the at least one symptom associated with the CRFR2 maladaptation is fatigue, pain, sleep issues, cognitive issues, orthostatic intolerance, body temperature perceptions, flu-like symptoms, headaches or sensory sensitivity, shortness of breath, gastrointestinal issues, urinary issues, musculoskeletal issues, nervous system issues, anxiety, depression, or other characterizations or manifestations of the foregoing.

13. The method of claim 11, wherein persistent improvement comprises improvement in the at least one symptom associated with the CRFR2 maladaptation for at least 1 week or longer following cessation of the administration of the CRFR2 agonist.

14. The method of claim 11, wherein the CRFR2 maladaptation is a functional somatic syndrome.

15. The method of claim 14, wherein the functional somatic syndrome is myalgic encephalomyelitis/chronic fatigue syndrome.

16. The method of claim 11, wherein the CRFR2 maladaptation is a post-acute sequelae of SARS-CoV-2 infection.

17. The method of claim 11, wherein the CRFR2 agonist is one or more of UCN1, UCN2, UCN3, SRP, SCP, CT38, CT37, or a pharmaceutically acceptable salt or solvate thereof.

18. The method of claim 11, wherein the CRFR2 agonist contains an acetate salt of CT38 (CT38s).

19. The method of claim 18, wherein the controlled-release dose of the CT38s is administered at a rate not exceeding about 0.03 μg/kg/h.

20. The method of claim 18, wherein the CT38s is administered to maintain the plasma concentration below about 0.25 ng/ml of CT38s to induce persistent improvement of the at least one symptom associated with the CRFR2 maladaptation.
